(19) United States
(12) Patent Application Publication
(10) Pub. No.: US 2008/0176255 A1
(43) Pub. Date: Jul. 24, 2008

(54) Mold detection composition and methods

(76) Inventor: Edward Sobek, Oak Ridge, TN (US)

Correspondence Address: Pearl Cohen Zedek Latzer, LLP, 1500 Broadway, 12th Floor, New York, NY, 10036, US

(21) Appl. No.: 11/656,556

(22) Filed: Jan. 23, 2007

(51) Int. Cl. G01N 33/533; C07C 317/12

(52) U.S. Cl. 435/7.31; 252/364; 252/586; 568/34

(57) ABSTRACT

This invention relates to compositions and methods for the detection of mold. Specifically, provided herein are compositions and methods for the detection of mold by detecting differences in light intensity of fluoresence resulting from interaction between the mold and the compositions described herein.

### FIELD OF THE INVENTION

This invention is directed to compositions and methods for the detection of mold. Specifically, provided herein are compositions and methods for the detection of mold by detecting differences in light intensity of fluoresence resulting from interaction between the mold and the compositions described herein.

### BACKGROUND OF THE INVENTION

Various fluorescent stains have been used to identify fungal particles in clinical and laboratory settings. However, the techniques and stains employed require extensive preparation, are slow acting, and are often ineffective at staining the various asexual and sexual spore types produced by ascomycetes, zygomycetes and basidiomycetes. These may comprise the vast majority of fungi having clinical, agricultural, and/or industrial application. Therefore, a need exists for a dye which is rapid acting for detecting such mold spores.

### SUMMARY OF THE INVENTION

In one embodiment, the invention provides a fluorescent staining composition for mold detection, comprising: 2,2′-([1,1′-Biphenyl]-4,4′-diyldi-2,1-ethenediyl)bis-benzenesulfonic acid disodium salt (BBD).

In another embodiment, provided herein is a method for producing a high-viscosity fluorescent staining composition for mold detection, comprising: mixing 2,2′-([1,1′-Biphenyl]-4,4′-diyldi-2,1-ethenediyl)bis-benzenesulfonic acid disodium salt (BBD), a gelling agent, a solvent, and a diluent together solubilizing the mixture in the diluent; and cooling the staining solution below the gelling temperature of the gelling agent.

In one embodiment, provided herein is a method for producing a low-viscosity fluorescent staining composition for a mold detection, comprising: mixing, 2,2′-([1,1′-Biphenyl]-4,4′-diyldi-2,1-ethenediyl)bis-benzenesulfonic acid disodium salt (BBD), a solvent, and a diluent togther; and solubilizing the mixture in the diluent.

In another embodiment, provided herein is a method of detecting a mold in solution comprising: contacting the solution with a dye comprising 2,2′-([1,1′-Biphenyl]-4,4′-diyldi-2,1-ethenediyl)bis-benzenesulfonic acid disodium salt (BBD), a solvent, and a diluent; exposing the contacted solution to electromagnetic radiation; and analyzing the difference in fluorescence between the contacted and uncontacted solution, whereby an increase in light intensity of fluorescence of above about 45% indicates the presence of mold.

In one embodiment, provided herein is a method of detecting a mold on a surface comprising the steps of: collecting a sample; contacting the sample with a dye comprising 2,2′-([1,1′-Biphenyl]-4,4′-diyldi-2,1-ethenediyl)bis-benzenesulfonic acid disodium salt (BBD), a gelling agent, a solvent, and a diluent; exposing the contacted sample to electromagnetic radiation; and analyzing the difference in fluorescence between the collected sample and the contacted sample, whereby an increase in the light intensity of fluorescence of above about 45% indicates the presence of mold.

In another embodiment, provided herein is a method of quantifying mold concentration in a sample, comprising: contacting the sample containing the mold with 2,2′-([1,1′-Biphenyl]-4,4′-diyldi-2,1-ethenediyl)bis-benzenesulfonic acid disodium salt (BBD); and comparing the observed light intensity of fluorescence to a standard.

The method and apparatus of the present invention will be better understood by reference to the following detailed discussion of specific embodiments and the attached figures which illustrate and exemplify such embodiments.

### BRIEF DESCRIPTION OF THE DRAWINGS

A specific embodiment of the present invention will be described with reference to the following drawings, wherein:

FIG. 1 shows Aspergillus niger stained spores on 13 mm glass fiber membranes imaged on a Kodak 4000MM imaging station. Membranes are the same membranes from which spore concentrations are reported in Table 1.0. From left to right spore concentrations are from highest to lowest the rightmost membrane is the control and is not visible. Spore concentrations (230,000 spore, 15,000 spores, 1800 spores, negative control).

### DETAILED DESCRIPTION OF THE INVENTION

This invention relates in one embodiment to compositions and methods for the detection of mold. Specifically, provided herein are compositions and methods for the detection of mold by detecting the light intensity of fluoresence resulting from interaction between the mold and the compositions are described herein.

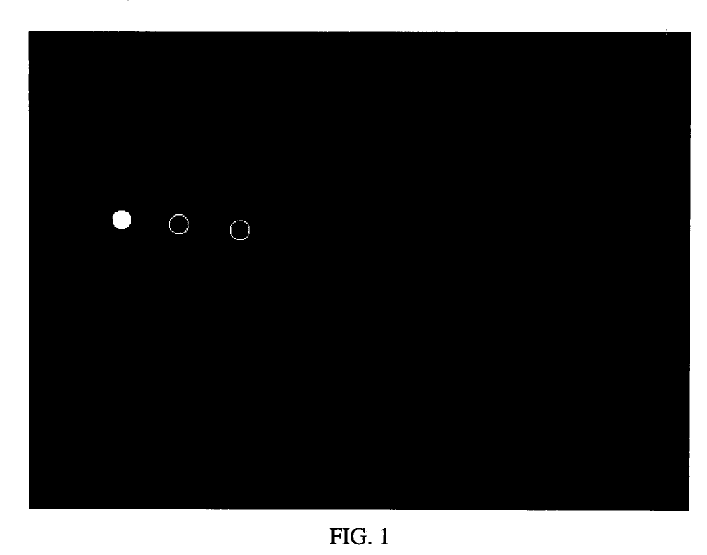

FIG. 1 shows an embodiment of the result of the methods of detection and quantification of mold as described hereinabove. Spores of known concentrations are quantified by a hemacytometer counting chamber under 40× magnification are shown. The fluorescence emission as shown in FIG. 1 ndicates the existence of mold. The intensity of the flourescence further indicates and quantifies the concentration of mold present.

In another embodiment, the novel fluorescent fungal staining gel described herein, may be used to capture and stain fungal spores and particles from the air by using a wide variety of spore collecting devices and pumps. Such spore collecting devices are well known to those skilled in the art. Moreover, the capture gel may allow for on-site or field quantification of fungal spores and particles. This may be accomplished by using standard detection devices outfitted with a light source, excitation/emission filters, and/or a fluorescent light detector.

According to one aspect of the invention and in one embodiment, a fluorescent staining composition for mold detection, may comprise: 2,2′-([1,1′-Biphenyl]-4,4′-diyldi-2,1-ethenediyl)bis-benzenesulfonic acid disodium salt (BBD). In another embodiment, 2,2′-([1,1′-Biphenyl]-4,4′-diyldi-2,1-ethenediyl)bis-benzenesulfonic acid disodium salt (BBD) is represented by the following structure:

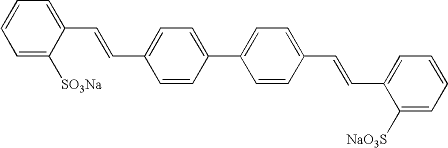

2,2′-([1,1′-Biphenyl]-4,4′-diyldi-2,1-ethenediyl)bis-benzenesulfonic acid disodium salt (BBD) is anionic and has a solubility in distilled water of 25 g/l at 25° C., and 300 g/l at 95° C. In one embodiment, the compositions described herein may further comprise a solvent; a gelling agent; and a diluent.

In one embodiment, the solvent used may be Dimethyl Sulfoxide (DMSO), absolute methanol, or other solvents capable of improving the availability of the dye in the compositions used herein. In one embodiment, the gelling agent used in the compositions described herein is glycerol, gellatine, agar, a synthetic hydrocolloid, or a natural hydrocolloid. It has been found that any appropriate material capable of increasing the viscosity of the composition may be used as a gelling agent. The increase in viscosity may be accomplished by the introduction of inert solids, or with any compound acquiring waters of hydration, or through chain entanglement. The gelling agent encompassed in the compositions described herein are those compounds that will delay the flow of the compositions off the samples or surfaces to which they are applied. In another embodiment, the diluent used is pyrogen-free water. Any compound that will increase the viscosity of the solution having the dye is encompassed by the term, “gelling agent”.

In one embodiment, the gelling agent used in conjunction with the methods and compositions provided herein is Alginic acid, or Sodium alginate, Potassium alginate, Calcium alginate, Agar, Carrageenan, Carob gum, Gelatine, Propylene glycol alginate, gum Arabic, Tragacanth, Guar gum, Xanthan gum, Karaya gum, Tara gum, Gellan gum, Polydextrose, Dextrin, Modified starch, Alkaline modified starch, Bleached starch, Oxidized starch, Monostarch phosphate, Distarch phosphate, Distarch phosphate, Phosphated distarch phosphate, Acetylated distarch phosphate, Acetylated starch mono starch acetate, Acetylated starch, Acetylated distarch adipate, Distarch glycerine, Hydroxy propyl starch, Hydroxy propyl distarch glycerine, Hydroxy propyl distarch phosphate, Starch sodium octenyl succinate, Acetylated oxidised starch, Polyvinyl Alcohol, Phytagel, Transfergel, and their combination in other embodiments.

In one embodiment, the mold sought to be detected or quantified using the compositions and methods described herein, is Stachybotrys chartarum, or Penicillium chrysogenum, Penicillium islandicum, Penicillium sp., Aspergillus niger, Rhizopus nigricans or any combination thereof. In other embodiments, other fungi species may be detected or quantified using the compositions and methods described herein. In another embodiment, the mold sought to be detected or quantified using the compositions and methods described herein, are listed in Table 3.0 provided hereinbelow.

TABLE 3.0
Fungi for detection/quantification

| | | |
|---|---|---|
| Absidia corymbifera | Aspergillus unguis | Eurotium repens |
| Acremonium strictum | Aspergillus ustus | Epicoccum nigrum |
| Alternaria alternata | Aspergillus versicolor | Fusarium solani |
| Aspergillus auricomus | Aspergillus wentii | Geotrichum candidum |
| Aspergillus caespitosus | Aureobasidium pullulans | Geotrichum klebahnii |
| Aspergillus candidus | Candida albicans | Memnoniella echinata |
| Aspergillus carbonarius | Candida dubliniensis | Mucor amphibiorum |
| Aspergillus cervinus | Candida glabrata | Mucor circinelloides |
| Aspergillus clavatus | Candida haemulonii | Mucor hiemalis |
| Aspergillus flavus | Candida krusei | Mucor indicus |
| Aspergillus giganteus | Candida lipolytica | Mucor mucedo |
| Aspergillus oryzae | Candida lusitaniae | Mucor racemosus |
| Aspergillus fumigatus | Candida maltosa | Mucor ramosissimus |
| Neosartorya fischeri | Candida parapsilosis | Rhizopus azygosporus |
| Aspergillus flavipes | Candida sojae | Rhizopus homothalicus |
| Aspergillus niger | Candida tropicalis | Rhizopus microsporus |
| Aspergillus awamori | Candida viswanathii | Rhizopus oligosporus |
| Aspergillus foetidus | Candida zeylanoides | Rhizopus oryzae |
| Aspergillus phoenicis | Chaetomium globosum | Myrothecium verrucaria |
| Aspergillus niveus | Cladosporiunm | Paecilomyces lilacinus |
| Aspergillus paradoxus | cladosporioides | Paecilomyces variotii |
| Aspergillus parasiticus | Cladosporium herbarum | Penicillium aethiopicum |
| Aspergillus sojae | Cladosporium | Penicillium |
| Aspergillus penicillioides | sphaerospermum | atramentosum |
| Aspergillus puniceus | Emericella nidulans | Penicillium |
| Aspergillus restrictus | Emericella rugulosa | aurantiogriseum |
| Aspergillus caesillus | Emericella quadrilineata | Penicillium freii |
| Aspergillus conicus | Emericella variecolor | Penicillium polonicum |
| Aspergillus sydowii | Eurotium amstelodami | Penicillium tricolor |
| Aspergillus sclerotiorum | Eurotium chevalieri | Penicillium viridicatum |
| Aspergillus tamarii | Eurotium herbariorum | Penicillium verrucosum |
| Aspergillus terreus | Eurotium rubrum | Scopulariopsis |
| Penicillium | Penicillium miczynskii | brevicaulis |
| brevicompactum | Penicillium olsonii | Scopulariopsis fusca |
| Penicillium stoloniferum | Penicillium oxalicum | Scopulariopsis brumptii |
| Penicillium canescens | Penicillium | Scopulariopsis chartarum |
| Penicillium chrysogenum | purpurogenum | Scopulariopsis |
| Penicillium griseofulvum | Penicillium restrictum | sphaerospora |
| Penicillium glandicola | Penicillium raisitrickii | Stachybotrys chartarum |
| Penicillium coprophilum | Penicillium roquefortii | Trichoderma asperellum |
| Penicillium expansum | Penicillium sclerotiorum | Trichoderma hamatum |
| Eupenicillium crustaceum | Penicillium | Trichoderma harzianum |
| Eupenicillium egyptiacum | simplicissimum | Trichoderma longibrachiatum |
| Penicillium verrucosum | Penicillium ochrochloron | Trichoderma citrinoviride |
| Penicillium variabilis | Penicillium lividum | Trichoderma viride |
| Penicillium thomii | Penicillium pupurescens | Trichoderma atroviride |
| Penicillium spinulosum | Penicillium spinulosum | Trichoderma koningii |
| Penicillium pupurescens | Rhizomucor meihei | Ulocladium botrytis |
| Penicillium lividum | Rhizomucor pusillus | Ulocladium chartarum |
| Penicillium islandicum | Rhizomucor variabilis | Wallemia sebi |
| Penicillium italicum | Rhizopus stolonifer | |
| Penicillium melinii | Scopulariopsis asperula | |

In one embodiment, the compositions for producing a high-viscosity fluorescent staining composition for mold detection or quantification, may comprise: mixing 2,2′-([1,1′-Biphenyl]-4,4′-diyldi-2,1-ethenediyl)bis-benzenesulfonic acid disodium salt (BBD), a gelling agent, a solvent, and a diluent together for solubilizing the mixture in the diluent; and cooling the staining solution below the gelling temperature of the gelling agent. In certain embodiments, whereby the gelling agent may be a solid, the step of cooling may not be necessary. Alternatively, cooling may be used and still be encompassed by the methods provided herein. As used herein, the term “high viscosity” refers to any composition where the measured viscosity is more than about 9 cP·s. Conversely, non-viscous compositions described herein, will have a viscosity of less than about 9 cP·s.

According to one embodiment, a method of producing a low-viscosity fluorescent staining composition for mold detection or quantification, may comprise mixing 2,2′-([1,1′-Biphenyl]-4,4′-diyldi-2,1-ethenediyl)bis-benzenesulfonic acid disodium salt (BBD), a solvent, and a diluent together; and solubilizing the mixture in the diluent.

In another embodiment, compositions made using the methods described above may be for detecting and/or quantifying mold or fungi. According to this aspect of the invention, a method of detecting a mold in solution may comprise contacting the solution with a dye comprising 2,2′-([1,1′-Biphenyl]-4,4′-diyldi-2,1-ethenediyl)bis-benzenesulfonic acid disodium salt (BBD), a solvent, and a diluent; exposing the contacted solution to electromagnetic radiation; and analyzing the difference in fluorescence intensity between the contacted and uncontacted solution, whereby an increase in light intensity of fluorescence of above about 45% indicates the presence of mold in the solution.

The fluorescence characteristics of the dyes described herein, are readily determined according to standard methods known in the art. In one embodiment, the excitation spectrum of the dye is determined by monitoring its emission at a constant wavelength while the excitation wavelength is varied, thereby generating a curve resembling an absorption spectrum. The emission spectrum of the dye may be determined by exciting the dye at a constant wavelength and analyzing the emitted spectrum. This may be done either directly or by analyzing the degree of transmission by a series of filters. The true spectra may be determined by normalizing for the wavelength-dependent intensity of the light source and the wavelength-dependent variation of the detector response. Such normalization is typically performed by comparing the detected spectra with corrected spectra of a known standard (e.g., quinine in sulfuric acid). In one embodiment, the excitation spectrum used in the methods provided herein is between about 330 nm and about 460 nm. In another embodiment, the excitation spectra may be between 350 nm and 455 nm. In still another embodiment, the excitation spectra may be between 360 nm and 400 nm. In a further embodiment, the excitation spectra may be between 360 nm and 480 nm. In a still further embodiment, the excitation spectra may be between 360 nm and 370 nm.

The term “fluorescent” refers to the property of a molecule which becomes excited and emits light of a longer wavelength or wavelengths upon irradiation with light of a given wavelength or wavelengths. The term “fluorophore” as used herein refers to a fluorescent molecule. There are a number of parameters which together describe the fluorescence characteristics of a fluorophore. These include, for example, the maximum wavelengths of excitation and emission, the breadth of the peaks for excitation and emission, the difference between the excitation and emission maxima (the “Stokes shift”), fluorescence intensity, quantum yield, and the extinction coefficient. For biological or biochemical applications, longer Stokes shifts may be generally preferred to shorter ones.

Fluorescence intensity may be determined as the product of the extinction coefficient and the fluorescence quantum yield. The fluorescence quantum yield is a measure of the relative efficiency or extent to which absorbed light energy is re-emitted as fluorescence. It is defined as the ratio of the number of fluorescence photons emitted, “F” to the number of photons absorbed, “A”. Molecules with larger quantum yields exhibit greater fluorescence intensity. The molar extinction coefficient is a measure of a fluorophore's ability to absorb light. Commonly used fluorophores tend to have molar extinction coefficients (at their absorption maximum) between 5,000 and 200,000 cm⁻¹ M⁻¹ (Haugland, R. P. (1996) Molecular Probes Handbook for Fluorescent Probes and Research Chemicals, 6th Edition). Higher extinction coefficients also correlate with greater fluorescence intensity because fluorescence intensity is the product of quantum yield and the extinction coefficient.

In one embodiment, the methods and compositions described hereinabove may be a whole or a part of the methods described herein. A method of detecting a mold on a surface may comprise the steps of collecting a sample; contacting the sample with a dye comprising 2,2′-([1,1′-Biphenyl]-4,4′-diyldi-2,1-ethenediyl)bis-benzenesulfonic acid disodium salt (BBD), a gelling agent, a solvent, and a diluent; exposing the contacted sample to electromagnetic radiation; and analyzing the difference in fluorescence intensity between the collected sample and the contacted sample. An increase in the light intensity of fluorescence of above about 45% may generally indicate the presence of mold. The fluorescent dye used uin the compositions and methods provided herein, reacts in one embodiment with spore wall glucans, carbohydrates and glycoproteins. The dye is rapidly bound to the aforementioned molecules. The binding is irreversible and permanent. Unlike most fluorescent dyes that “wash-out” or fade with exposure to fluorescent light at the excitation peak, the dyes described herein is stable for at least 6 months when stored at room temperature and years in other embodiments if refrigerated and kept in the dark.

In one embodiment, detection and quantification of the mold or fungi using the methods and compositions provided herein is done using Fluorescent microscopes (e.g. Leica 5500), or plate readers (e.g. Tecan Infinite M200), imaging systems (e.g. Kodak Imaging Station 4000MM), Quantitative PCR machines (e.g. Corbett RotoGene 6000), or their combination in other embodiments.

Collecting a sample may comprise any collection method now known in the art or developed in the future, so long as it lends itself to be contacted with the compositions described herein. These methods include but are not limited to swabbing, vaccuum-assisted, wash-away, wiping, filtering, and the like as is well known to those skilled in the art.

In one embodiment, several fluorescence readouts may be used for quantifying a mold's intensity, anisotropy, or spectral characteristics. In other embodiments, a variety of measurement techniques may be used including, for example, confocal microscopy, flow cytometry, and the like.

In another embodiment, the methods may be used for quantifying mold in a sample. According to this aspect, the method may comprise contacting the sample containing the mold with 2,2′-([1,1′-Biphenyl]-4,4′-diyldi-2,1-ethenediyl)bis-benzenesulfonic acid disodium salt (BBD); and comparing the observed light intensity of fluorescence to a standard. In one embodiment, the standard may be obtained from a sample containing a known concentration. In another embodiment, the standard may be a range of concentrations in solution together with their accompanying light intensity.

In one embodiment, a solution comprising the compositions provided herein is directly applied to a surface, in a field setting, and thereafter, exposed to electromagnetic radiation in order determine the presence and quantity of mold. Detection is done either by a direct visual examination with the naked eye in one embodiment, or viewed by the naked eye through a emission filter (e.g. glasses or goggles), or detected with a electromagnetic radiation detector in other embodiments.

In still another embodiment, a kit may be used for detecting or quantifying mold in a sample comprising the compositions described herein. Instructions, packaging materials, and standards may also be included in such a kit. Optimal excitation wavelengths and the expected shift when a given mold is reacted or contacted with the compositions described herein may be included as part of the instructions. Such kits may be adapted for a specific mold, which may be Stachybotrys chartarum in one embodiment, or Penicillium chrysogenum, Penicillium islandicum, Penicillium sp., Aspergillus niger, or Rhizopus nigricans, for example. Such kits may also comprise concentration-intensity curves that were obtained using the methods described herein.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented to illustrate preferred embodiments of the invention. It is to be understood that the following example of the present invention is not intended to restrict the present invention since many more modifications may be made within the scope of the claims without departing from the spirit thereof.

Materials and Methods
1. 2,2′-([1,1′-Biphenyl]-4,4′-diyldi-2,1-ethenediyl)bis-benzenesulfonic acid disodium salt (BBD)
2. Pyrogen free MilliQ water (in house Millipore water purification system)
3. Dimethyl Sulfoxide molecular biology grade (DMSO)
4. Glycerol 99.5%
5. Glass Fiber Membrane (Pall Corporation)
6. Hotplate Stirrer (VWR Scientific)
7. 150 ml glass beakers (VWR Scientific)
8. Type J thermocoupler
9. Infrared thermometer
10. 2.0 ml PCR Tubes (VWR)
11. foam tipped swabs (VWR)
12. hemacytometer counting chamber

Detection Systems For Testing Captured Spores

### Photomultiplier Tube System

1. UV Source: Light Emitting Diode (LED)
Center: 365 nm
Max Tail: 400 nm
Min Tail: 330 nm
Excitation Filter (UV Bandpass):

2. Peak Transmission: 360 nm
High Cut-off: 385 nm
Emission Filter (VIS Longpass):
Cut-off: 455 nm
Low Stop: 410 nm

Note: The desired wavelength reaching the detector from the fluorescing sample should be larger than about 430.

### CCD Based Detection System (Kodak 4000MM Molecular Imaging Station)

1. UV Source: Halogen 100 watt fiber optic system

2. Excitation Filter: ex385 (Kodak Cat#1829308)

3. Emission Filter: em440WA (Kodak Cat#8739518)

Fluorescent Microscope (Riechert MicroStar IV with DAPI Cluster K2834)

1. UV Source: Metal Halide Lamp 50 watt

2. Excitation: 365 nm

3. Emission: 390 nm

4. Dichloric Mirror: 395 nm

### Fungal Cultures

1. Stachybotrys chartarum

2. Penicillium chrysogenum

3. Penicillium islandicum

4. Penicillium sp.

5. Aspergillus niger

Methods

A variety of dye formulations were tested, but the following formulation has given the best results to date

### A. Gel Formulation

1. 0.01% BBD w/v

2. 25% Glycerol v/v

3. 0.1% DMSO

4. Pyrogen Free MilliQ water as solvent base

### B. Non-viscous Formulation

1. 0.01% BBD w/v

2. 0.1% DMSO

3. Pyrogen Free MilliQ water as solvent base

### EXAMPLE 1

### Dye Preparation Process

All glassware was baked at 200° C. for 2 hours to ensure that no fungal spores would contaminate the experiments and all plastic disposables were certified sterile.
a) 50 ml of pyrogen free MilliQ water was added to a 150 ml beaker containing a magnetic stir bar.
b) The beaker was placed onto a hot/plate stirrer and set to 40° C. at 250 rpm.
c) A thermocouple was inserted into beaker and attached to a infrared thermometer.
d) The chemicals for the dye formation were added to the 50 ml of H₂O based on an endpoint solvent solution of 100 ml, after the water reached 40° C.
e) The dye solution was allow to mix for 30 minutes or until all chemicals went into solution.
f) The dye solution was decanted into a 100 ml volumetric flask and the flask was brought to volume with the addition of MilliQ water.
g) The dye solution was briefly mixed in the volumetric flask and decanted back into the 150 ml beaker and place back onto the hotplate and stirred without heating for 10 minutes.
h) Afterwards 10 ml aliquots of the dye solution were transferred into 15 ml conical bottom tubes (VWR).
i) The tubes were stored in the dark at room temperature until use.

### EXAMPLE 2

### Various Methods Used for Testing Spore Stain

A. Direct spore release and capture using the SRCT system.
a) A 60 mm sterile Petri dish was filled with 5.0 ml of the Gel formulated dye.
b) Individual Glass fiber filter membranes were dipped into the solution and transferred to a sterile 100 mm Petri dish and allowed to dry overnight.
c) The test fungal cultures were grown for 5 days at 27° C. in the dark.
d) All cultures were producing large quantities of spores at the time of testing.
e) A 5 mm plug of the test culture was removed and placed into a home-made spore-release and capture tube (SRCT).
f) The SRCT is extremely versatile and can be connected to a variety of air collection devices and spore traps for the purposes of testing spore capture efficacy.
g) The fungal culture plug was placed in one end of the tube, and the other end was connected to a sampling cassette containing the Gel-coated membrane, which in turn was connected to a vacuum pump.
h) The vacuum pump was set to pull at 5 liters/minute.
i) When started, the pump creates an air stream that moves across the plug of fungus, removing spores and pulling them directly onto the gel-coated membrane.
j) Samples were collected for 1 minute to load ˜10,000 spores onto the gel coated membrane.
k) Time and pump volume rate can be varied to deliver more or less spores depending on the particular application.

B. Direct Application/Quick Wash (Table 1.0 and FIG. 1.0)
a. A solution of the non-viscous dye formulation was pipetted into 2.0 ml sterile PCR tubes.
b. Various quantities of spores were added to the solution using sterile foam tipped swab and 5 day old sporulating fungal cultures.
c. the PCR tube was mixed using a vortex for 30 seconds
d. 25 ul of the spore suspension was immediately deposited directly onto the glass fiber membrane.
e. The membrane was mounted on a VWR funnel glass filtration unit, which was connected via a rubber stopper to a 1000 ml Erlenmeyer side-arm flask.
f. The membrane was washed with 20 ml of MilliQ water via filtration.
g. The membrane and spore were immediately examined via the photomultiplier tube system followed by the CCD system.
h. An aliquot of the spores from the various concentrates was quantified in a hemacytometer counting chamber under 40× magnification.

TABLE 1.0
Aspergillus niger Stained Spores detected with Photomultiplier Tube System

| | | Filter | Background | Spores | Percentage Increase in |
|---|---|---|---|---|---|
| | Ambient | Only | Dye | Stained | Fluorescence Signal |
| Control Filter | no spores | 0.18 | 0.67 | NA | |
| Spore Conc. 1 | 1800 Spores | 1.15 | 1.1 | 1.64 | 49.09% |
| Spore conc. 2 | 15,000 Spores | 1.25 | 1.07 | 2.76 | 157.94% |
| Spore Conc. 3 | 230,000 Spores | 1.15 | 1.15 | 4.46 | 287.83% |

Direct Fluorescent Microscopic examination of Gel Formulation (Table 2.0)

In a separate experiment spores were examined under a fluorescent microscope to confirm that they were indeed adsorbing and emitting a fluorescent signal.
1. A surface swab was collected from a sporulation colony of fungus.
2. The swab was mixed with 1.0 ml of the Gel-dye formulation.
3. The solution was allowed to incubate for 30 minutes at 27° C.
4. A aliquote of the solution was mounted on glass slides and examined under the fluorescent microscope to determine the effectiveness of the Gel-dye formulation.

TABLE 2.0
Fluorescent Microscopic examination of Gel stained fungal spores
and mycelium.

Stain Intensity

| | Stained | |
|---|---|---|
| Fungal Culture | Mycelium | Stained Spores |
| Stachybotrys chartarum | bright | bright |
| Penicillium chrysogenum | bright | dim |
| Penicillium islandicum | bright | dim |
| Unknown Penicillium sp. | bright | dim |
| Aspergillus niger | bright | bright |

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A fluorescent staining composition for mold detection, comprising: 2,2′-([1,1′-Biphenyl]-4,4′-diyldi-2,1-ethenediyl)bis-benzenesulfonic acid disodium salt (BBD).

2. The composition of claim 1, further comprising a solvent; a gelling agent; and a diluent.

3. The composition of claim 2, wherein the solvent is Dimethyl Sulfoxide (DMSO)

4. The composition of claim 2, wherein the gelling agent is glycerol, gellatine, agar, a synthetic hydrocolloid; or a natural hydrocolloid.

5. The composition of claim 2, wherein the diluent is pyrogen free water.

6. The composition of claim 1, wherein the mold is one of the mold species described in table 3.

7. A method for producing a high-viscosity fluorescent staining composition for mold detection, comprising:
a. mixing 2,2′-([1,1′-Biphenyl]-4,4′-diyldi-2,1-ethenediyl)bis-benzenesulfonic acid disodium salt (BBD), a gelling agent, a solvent, and a diluent together
b. solubilizing the mixture in the diluent; and
c. cooling the staining solution below the gelling temperature of the gelling agent.

8. The method of claim 7, whereby wherein the solvent is Dimethyl Sulfoxide (DMSO)

9. The method of claim 7, wherein the gelling agent is glycerol, gellatine, agar, a synthetic hydrocolloid, or a natural hydrocolloid.

10. The method of claim 7, wherein the diluent is pyrogen free water.

11. The method of claim 7, wherein the mold is one of the mold species described in table 3.

12. A method for producing a low-viscosity fluorescent staining composition for a mold detection, comprising:
a. mixing 2,2′-([1,1′-Biphenyl]-4,4′-diyldi-2,1-ethenediyl)bis-benzenesulfonic acid disodium salt (BBD), a solvent, and a diluent togther; and
b. solubilizing the mixture in the diluent.

13. The method of claim 12, whereby the solvent is Dimethyl Sulfoxide (DMSO)

14. The method of claim 12, whereby the diluent is pyrogen free water.

15. The method of claim 12, whereby the mold is one of the mold species described in table 3.

16. A method of detecting a mold in solution comprising:
a. contacting the solution with a dye comprising 2,2′-([1,1′-Biphenyl]-4,4′-diyldi-2,1-ethenediyl)bis-benzenesulfonic acid disodium salt (BBD), a solvent, and a diluent;
b. exposing the contacted solution to electromagnetic radiation; and
c. analyzing the difference in fluorescence intensity between the contacted and uncontacted solution, whereby an increase in light intensity of fluorescence of above about 45% indicates the presence of mold.

17. The method of claim 16, whereby the solvent is Dimethyl Sulfoxide (DMSO)

18. The method of claim 12, whereby the diluent is pyrogen free water.

19. The method of claim 12, whereby the mold is one of the mold species described in table 3.

20. A method of detecting a mold on a surface comprising the steps of:
a. collecting a sample;
b. contacting the sample with a dye comprising 2,2′-([1,1′-Biphenyl]-4,4′-diyldi-2,1-ethenediyl)bis-benzenesulfonic acid disodium salt (BBD), a gelling agent, a solvent, and a diluent;
c. exposing the contacted sample to electromagnetic radiation; and
d. analyzing the difference in fluorescence intensity between the collected sample and the contacted sample, whereby an increase in the light intensity of fluorescence of above about 45% indicates the presence of mold.

21. The method of claim 20, whereby the solvent is Dimethyl Sulfoxide (DMSO).

22. The method of claim 20, wherein the gelling agent is glycerol, gellatine, agar, a synthetic hydrocolloid; or a natural hydrocolloid.

23. The method of claim 20, whereby the diluent is pyrogen free water.

24. The method of claim 20, whereby the mold is one of the mold species described in table 3.

25. A method of quantifying mold concentration in a sample, comprising:
a. contacting the sample containing the mold with 2,2′-([1,1′-Biphenyl]-4,4′-diyldi-2,1-ethenediyl)bis-benzenesulfonic acid disodium salt (BBD); and
b. comparing the observed light intensity of fluorescence to a standard.

26. The method of claim 25, whereby the mold is one of the mold species described in table 3.

27. The method of claim 26, whereby the standard is obtained from a sample containing a known concentration.

28. A kit for detecting, quantifying or both of mold, comprising: 2,2′-([1,1′-Biphenyl]-4,4′-diyldi-2,1-ethenediyl)bis-benzenesulfonic acid disodium salt (BBD), packaging material and instructions for detecting, quantifying or both of mold.

29. The kit of claim 28, further comprising a solvent; a gelling agent; and a diluent.

30. The kit of claim 28, further comprising known a mold standard.

31. The kit of claim 30, wherein the mold is one of the mold species described in table 3.

32. The kit of claim 30, wherein the standard comprises a series of known mold concentration in a predetermined solution.
